(19) United States
(12) Patent Application Publication
(10) Pub. No.: US 2024/0306968 A1
(43) Pub. Date: Sep. 19, 2024

(54) SYSTEM AND METHOD FOR STIMULUS FEEDBACK INTERVENTION

(71) Applicant: Purdue Research Foundation, West Lafayette, IN (US)

(72) Inventors: Nathan Govindarajan, Plano, TX (US); Peter Arthur Zoss, Lafayette, IN (US); Matthew Peter Ward, Zionsville, IN (US)

(73) Assignee: Purdue Research Foundation, West Lafayette, IN (US)

(21) Appl. No.: 18/608,768

(22) Filed: Mar. 18, 2024

Related U.S. Application Data
(60) Provisional application No. 63/452,524, filed on Mar. 16, 2023.

(51) Int. Cl. A61B 5/16; A61B 5/00; A61B 5/0205; A61B 5/021; A61B 5/0245; A61B 5/11; A61B 5/145

(52) U.S. Cl. CPC A61B 5/165; A61B 5/002; A61B 5/0022; A61B 5/0024; A61B 5/0205; A61B 5/4035; A61B 5/6803; A61B 5/7275; A61B 5/742; A61B 5/021; A61B 5/0245; A61B 5/1107; A61B 5/1112; A61B 5/14532; A61B 5/14542; A61B 5/4266; A61B 5/4809; A61B 5/4866; A61B 2560/0242

(57) ABSTRACT

A system for integrating a plurality of biosensor devices includes one or more communication interfaces adapted to communicate with each of the plurality of biosensor devices connected to a subject, and a processor executing software on a non-transitory memory to: establish a communication link with each of the plurality of biosensor devices, receive biodata from each of the plurality of biosensor devices through the one or more communication interfaces at a sampling rate, assign a timestamp to the received biodata such that the received data from the plurality of biosensor devices are synchronized, receive environmental data via one or more environmental sensors, store received biodata along with the assigned timestamp into a database and the environmental data, analyze stored data along with the assigned timestamp to predict an evoked response to one or more stimuli and the environmental data and provide a feedback based on the analysis.

### CROSS-REFERENCE TO RELATED APPLICATIONS

This patent application claims priority to a U.S. provisional patent application Ser. No. 63/452,524 filed Mar. 16, 2023, contents of which are hereby incorporated by reference in its entirety into the present disclosure.

### STATEMENT REGARDING GOVERNMENT FUNDING

None.

### TECHNICAL FIELD

The present disclosure generally relates to sensing responses from a subject and in response thereto analyze the response and provide feedback therefor.

### BACKGROUND

This section introduces aspects that may help facilitate a better understanding of the disclosure. Accordingly, these statements are to be read in this light and are not to be understood as admissions about what is or is not prior art.

Mental health disorders such as major depression and generalized anxiety disorder are often associated with impaired or abnormal autonomic regulation of organ/tissue physiology. This impaired or abnormal regulation of physiological activity (e.g., neural, endocrine, humoral) can be measured directly from neural substrates (e.g., from specific brain centers like the medial prefrontal cortex or from nerves like the vagus nerve), changes in blood chemistry, or changes in immune function (changes in behavior can be measured through a variety of psychological evaluations). These invasive methods are impractical for everyday use (especially for therapeutic applications), so recent research and development efforts have focused on measuring normal/abnormal physiological functions using wearable digital health technology like smart watches, which have a variety of sensors that measure analogs of organ or organ system physiology.

The nervous system mediates our body's involuntary and voluntary responses to endogenous or exogenous stimuli (herein referred to as environmental stimuli), which differ in time, circumstance and by individual in healthy or diseased states. The system response to environmental stimuli can be measured and inferred from various biosignals that describe the reflexive, involuntary functions of the autonomic nervous system (ANS) and to a lesser extent other systems (described below). However, this type of information is not well incorporated into current technology and interventions used in practice. An impaired mental state can manifest as abnormal autonomic regulation of thoracic (e.g., heart, lungs, immune tissues throughout the body), abdominal (e.g., stomach, liver, pancreas, spleen), or pelvic organ (e.g., bladder) functions, with differing abnormalities across short (e.g., seconds to minutes) and long (e.g., months to years) timeframes. Common examples of an impaired mental state are the different types of anxiety that afflict a large portion of the population; anxiety disorders are only one subset of the applications and is not the only use case for the enclosed system. Anxiety comes in many forms with many symptoms and behavioral outcomes, but to better understand treatment options for anxiety, it is also important to know how the pathophysiology governs the body's functional response to anxiety and stress. The ANS is a component of the peripheral nervous system and responsible for coordinating physiological processes that help maintain a healthy balance (e.g., respond to, restore, and/or maintain homeostasis in response to endogenous or exogenous stimuli, such as stressful social encounters, bacterial/viral infection or injury) within the body from the level of cells to organ systems. While the response to stress in our body is primarily controlled by the ANS, important roles are also played by the sympathetic-adreno-medullary axis (SAM-responsible for adaptive responses to stress within seconds of a stressful stimulus) and the hypothalamic-pituitary-adrenal (HPA axis-responsible for adaptive and sometimes maladaptive responses to stress on time scales ranging from minutes to hours). The ANS comprises three divisions: the sympathetic nervous system (SNS), the parasympathetic nervous system (PNS), and the enteric nervous system (ENS).

Besides anxiety, there a number of other mental and health ailments that can benefit from technology based on understanding of the ANS (especially the SNS and PNS divisions) and stress-response systems. Suppose a subject is suffering from alcoholism. While the subject may be equipped with various tools provided by his/her therapist to overcome craving/urges, many times situational awareness is lacking, and the subject is confronted by unanticipated circumstances that can result in severe and unfortunate cravings that increase odds of relapse. These context-dependent cravings and the desire to avoid consuming alcohol is an intense stressor, one that could be measured/detected from a pattern of evoked responses that manifest as specific sequences and levels of changes in ANS activity. A critical prior limitation of trying to understand internal motivations/drives from ANS activity is not being able to reliably attribute a particular physiological response to a particular cause. The ambiguity of this autonomic reactivity to the environmental stressor can be overcome when paired with contextual information (e.g., environmental stimuli that are perceived with our innate senses, e.g., sight, sound, smell, taste, and other contextual factors). In other words, one can attribute the type of autonomic reactivity to a particular cause with greater specificity if understood within the context in which it occurred. This contextual data immediately makes the information contained with the autonomic reactivity/response profile more valuable to treatment providers and patients alike. To the best of our knowledge, no technology has been previously developed to map autonomic reactivity (e.g., to specific environmental stimuli) to the perceived subjective/individual experience(s) within that environment. Furthermore, no such technology has been developed to serve as a conduit to individualized treatments.

Therefore, there is an unmet need for a novel method and system that can use technology to sense specific responses from a subject and provide feedback to the subject in order to assist to prevent undesirable outcomes.

### SUMMARY

A system for integrating a plurality of biosensor devices is disclosed. The system includes a plurality of biosensor devices connected to a subject. The system further includes one or more communication interfaces adapted to communicate with each of the plurality of biosensor devices. Additionally the system includes a processor executing software on a non-transitory memory. The execution of the software configures the processor to establish a communication link with each of the plurality of biosensor devices each through the one or more communication interfaces, receive biodata from each of the plurality of biosensor devices through the one or more communication interfaces at a sampling rate, assign a timestamp to the received biodata such that the received data from the plurality of biosensor devices are synchronized, receive environmental data via one or more environmental sensors, store received biodata along with the assigned timestamp into a database and the environmental data, analyze stored data along with the assigned timestamp to predict an evoked response to one or more stimuli and the environmental data, and provide a feedback to the subject or a healthcare worker based on the analysis.

### BRIEF DESCRIPTION OF DRAWINGS

FIG. 1 is a control flow of how a general embodiment of the apparatus of the present disclosure integrates multiple data streams under a common timebase.

FIG. 2 is a control flow for a variety of different devices according to the present disclosure.

FIG. 3 is an illustrative schematic showing how evenly demarcated timestamps are generated.

FIG. 4 is a generic network schematic showing a network of communication between the system of the present disclosure and various devices.

FIG. 5 is a flowchart of a general collector control flow that can be implemented to collect samples and timestamps, according to the present disclosure.

FIG. 6 is a flowchart depicting the control flow for the Varjo XR-3 headset to collect samples and timestamps, according to the present disclosure . . .

FIG. 7 is a flowchart depicting the control flow of how the heat map works in Unity for the Varjo XR-3 headset, according to the present disclosure . . .

FIG. 8 is a flowchart depicting a collector layer, according to one embodiment, for any device that interfaces with the Labchart server to collect samples, according to the present disclosure . . .

FIG. 9 is another schematic showing how devices are networked in the system of the present disclosure.

FIG. 10 is table showing sample entries into the implemented embodiment's database InfluxDB.

FIG. 11 is a heatmap depicting the augmented reality view inside the Varjo XR-3 headset with the heatmap tracking where the subject is looking at and gaze time(s), also implementable in virtual reality.

FIGS. 12a, 12b, and 12c are descriptive overview flowcharts of the different types of biosignals referred to herein and how the data associated with these bio-signals are transferred from the biosensors (e.g., wearables, etc.) to a central database and finally to a point where they can be used in a personalized model (referred to as the “Autonomic Reactivity Profile,” or ARP).

FIGS. 13a, 13b, 13c, 13d, and 13e are flowcharts spanning across 5 pages. The flowchart in these figures provide a high-level process flow that describes the model training sequence and therapeutic/treatment sequence for a generalized application of the present system and method to treat anxiety disorders, substance use disorder(s), etc.

FIG. 14 is a block diagram of a computer system that can interface with the system of the present disclosure.

### DETAILED DESCRIPTION

For the purposes of promoting an understanding of the principles in the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

A novel method and system are disclosed that can use technology to sense specific responses from a subject and provide feedback to the subject in order to assist to prevent undesirable outcomes. Towards this end, a system is disclosed herein capable of integrating in real time signals and data from a variety of sources that can provide biosignals from a subject. These signals and data are integrated into an analyzable package of data, the package analyzed, and feedback to the user or a healthcare worker is then generated based on the analysis.

To diagnose context-dependent anxiety, access to a variety of autonomic biosignals is necessary. Biosignals required can come from an array of different devices (for example a smartwatch, a virtual reality (VR) headset, smart glasses, and other smart devices providing inputs to a user). Therefore, a system that can accept input in real time from all these devices is necessary. Furthermore, to be able to make a decision that correlates all available biosignals, they must all be under a common timebase and considered within context/environment, so at any point in time, an analysis can be made about all the biosignals. Therefore, the system of the present disclosure can collect and stream data in near real time from multiple data sources and package the input data into one place under a common time base for analysis, interpretation, and feedback.

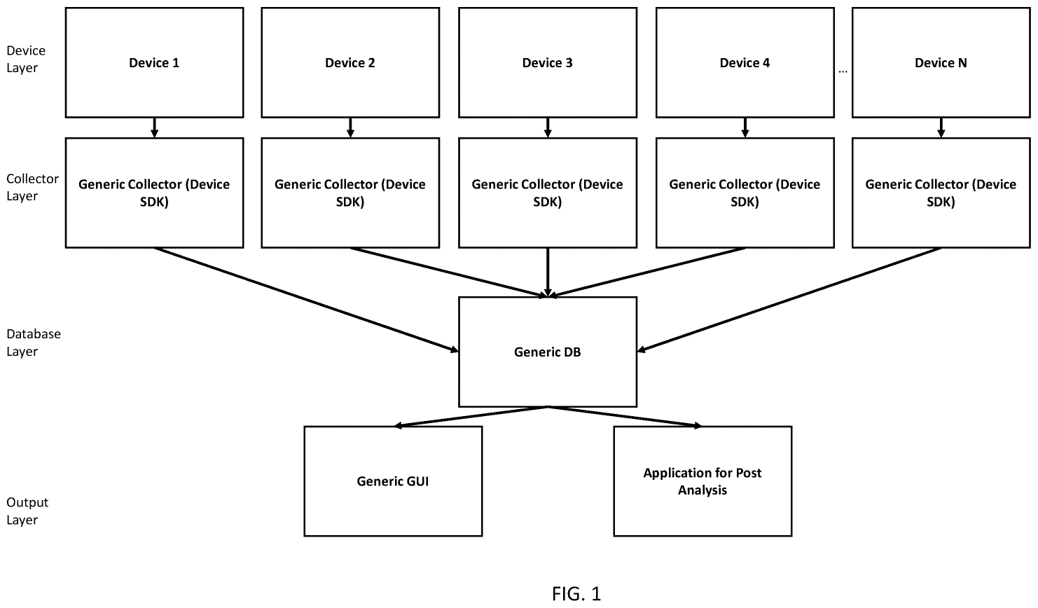

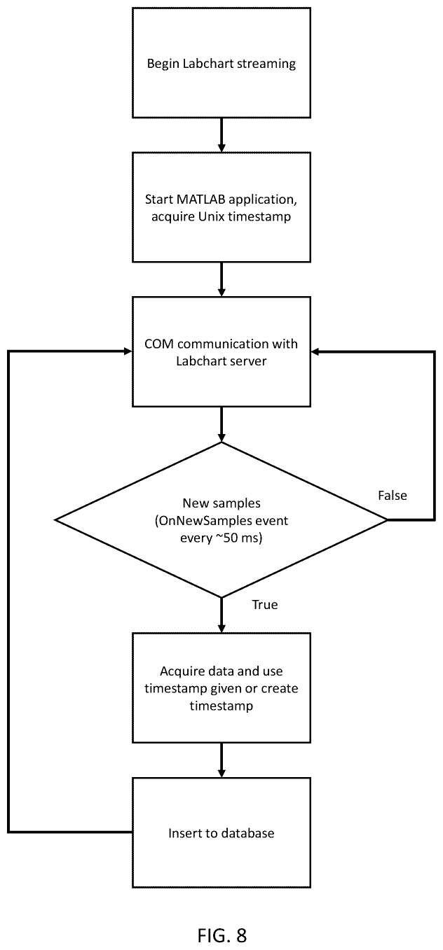

In the present disclosure, central processing unit (CPU), machine, and computer are used interchangeably, while data source is used interchangeably with device, device layer, and any device that collects biosignals from a subject. FIG. 1 is a control flow of how a general embodiment of the apparatus of the present disclosure integrates multiple data streams under a common timebase. Shown in FIG. 1 are four layers: 1) Device layer; 2) Collector layer (an example is shown in FIG. 8); 3) Database layer; and 4) Output layer. The device layer refers to devices that are responsible for ascertaining data to be collected. The collector layer refers to collector software applications which are developed using a device software development kit (SDK), or other toolkits known to a person having ordinary skill in the art, to programmatically access samples. For the database layer, a given timestamp can be used or alternatively one can be generated with evenly demarcated timestamps based on a predetermined sampling rate. Thus for the database layer, the timestamp, source type, and data to be stored in a database are provided. As a result, the database collects and stores the packet of information from multiple data sources. Data is analyzed and in the output layer, the user has the option to display the streaming results from said analysis and/or create an application for post analysis based on the data stored in the database. Biosignals can be sourced from any number of devices provided the device has an access point where data can be accessed. Processing power may become one bottleneck because collector applications need to be running simultaneously to collect data simultaneously. To alleviate this issue, a direct memory access topology, known to a person having ordinary skill in the art, may be implemented to avoid any processor bottleneck during data collection phase. In specific implementations, on average, a collector SDK may require 5-10% of CPU usage, when direct memory access is not utilized. Higher sampling rates may lead to higher CPU usage. Alternatively, it may be beneficial to spread out different collectors to run on different CPUs to minimize high CPU usage on any singular computer. Different machines may be used assuming timestamps are consistent and accurate across machines (e.g., Unix time with Network Time Protocol (NTP) or Precision Time Protocol (PTP) are protocols known to a person having ordinary skill in the art which can synchronize timestamps). The source of this challenge is that the database interacts via wireless Hypertext Transfer Protocol (HTTP) communication and only stores timestamp, source type, and data values, thus the database entry has no relation to the hardware the collector is associated with. As a result, collector applications can also be on any operating system.

In general, because the database communicates wirelessly with the various devices and is not on any specific machine, as mentioned above, any number of computers can interface (read and write) with the database. Thus, there can be multiple computers in the collection layer, and multiple computers can be utilized for the output layer.

In another implementation, multiple devices can stream data at separate times or at the same time. When multiple devices are streaming at the same time, the database needs an identifier to know which data belongs to which device as well as the timestamp, and data itself. Additionally, the database needs to maintain corresponding bibliographical information about the subject, e.g., the subject's name, etc.

The system of the present disclosure, thus, integrates a number of devices that have no connection between them, collect data simultaneously from said devices, and collects the data in near real time. The devices are sourced from different manufacturers and therefore have no means of directly interacting with each other, but for the central collection and analysis point of the present system.

As discussed above, any number of devices can be used provided the device has an SDK allowing access to data in the form of samples to be transferred. For example, according to the system of the present disclosure, a collector is provided for the Varjo XR-3 headset (a virtual reality headset) using the SDK supplied by VARJO. Accordingly, the system can either use a predetermined timestamp or generate evenly demarcated timestamps based on the predetermined sample/data acquisition rate. Next, the system sends the timestamp, source type, and data to the database, e.g., InfluxDB. InfluxDB expects timestamps in the form of Unix time in nanoseconds. The computer used is synchronized with the atomic clock in Boulder, Colorado using network time protocol (NTP). Data is then communicated using an HTTP POST request. InfluxDB receives and stores the data packets of information from all data sources, simultaneously. Finally, for the output layer, the user has the option to display the streaming results. Here, Grafana is used as a graphical user interface (GUI) to show streams because of its close relation with InfluxDB. The GUI uses structured query language (SQL). For example, to stream a specific source, the WHERE clause is used. An example query is: SELECT “value” FROM “dataStreams” WHERE (“source”=‘Pulse’) AND $timeFilter GROUP BY time ($_interval), “source”. The system also provides the option to analyze the data stored in the database in a post-processing manner.

In general, a collector should be an application (App) that runs on its own. While the App is running, the system may acquire available samples and timestamp if accessible. If a timestamp is not accessible, the system can create it based on new samples and the sample rate. The timestamp, data, and source are then all sent to a database, and the process repeats.

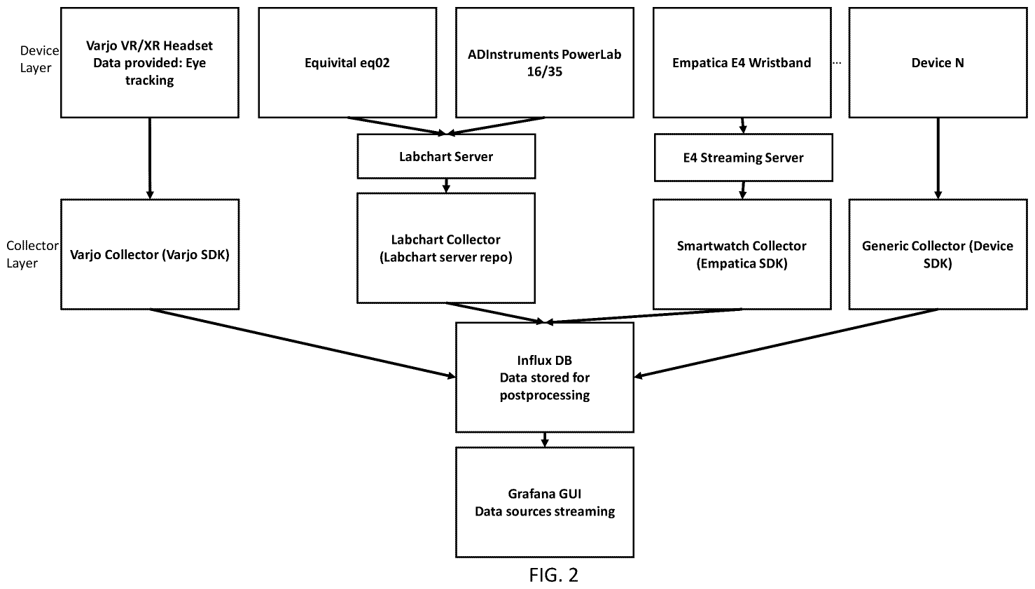

Referring to FIG. 2, a control flow for a variety of different devices is presented. The Varjo collector application utilizes the SDK supplied by Varjo to interact with the XR-3 headset. The Varjo system uses its own unit of time called “Varjo_nanoseconds”. Therefore, a synchronization between Unix time and Varjo time occurs by the system so that when samples are acquired, conversion to Unix time can occur.

The overall process is acquiring samples and filling a circular buffer with samples until it is full, where the buffer is then inserted into InfluxDB. Varjo XR-3 headset provides eye tracking (e.g., eye movement, pupil diameter, gaze time) at two sampling frequencies: 100 Hz or 200 Hz. Multithreading is used in this application to avoid any delays and avoid missing samples. Two threads are created: one thread focuses on acquiring the samples and writing to the buffer; the other thread continuously checks if the buffer is full and when it is, it communicates the source, timestamp, and value into the database. Timestamps in this collector are given by the SDK, and conversion from Varjo_nanoseconds to Unix time can then occur. Both threads continuously loop until the program is terminated.

The heat map in Unity works in conjunction with the Varjo XR-3 and the Varjo Unity SDK, but is translatable to other virtual, augmented, or mixed reality applications. The goal is to contextualize the change(s) in biosignals to what the subject was looking or experiencing before, during, and immediately after the change(s) in biosignals. The heat map collector application can be run at the same time as other collector applications. After clicking play in Unity, the user views images in the VR headset for instance, but this could also be done through observations of the real world environment in augmented reality (AR) mode as well. For an example relating to but not limited to the study of anxiety, these images are randomized and are intended to evoke an autonomic response by causing psychological stress/anxiety. While this is happening, the VR headset is tracking where the user is looking, so if an autonomic response is detected, upon analysis, the context of where the user was looking is thereby associated. The control flow is as follows. Every frame, an update function gets called which queries from the Varjo Unity SDK where the gaze is located. Then, that value is used to add a fixed value to a cell in a grid. The values can be added to 100 in this example. The higher the value of the cell in a grid, the different the color is. In this implementation, red corresponds to a lower number and green corresponds to a higher number. Therefore, if a subject were to stare at a single spot for an extended period of time, values in that specific cell would rise, and the spot shifts from red to green. For every frame, coordinates of the gaze location are sent to InfluxDB for post analysis purposes. Furthermore, because the user is viewing the heat map in virtual reality/augmented reality, any scenario can be created to stimulate evoked responses.

It should be understood that a variety of devices including VR headsets, smart glasses, and artificial intelligent devices are within the ambit of the present disclosure. These devices can be configured to provide a combination of virtual reality and actual reality in the form of images and video from the user's surrounding together.

For ADInstruments-derived biosignals, a GitHub repository is utilized that aids in connecting to the active Labchart document and therefore the Labchart server. The only requirement is that Labchart and its associated collector are running on the same computer. Next, the system acquires the Unix time at the start of the application as a reference point, enabling the system to convert all future timestamps to Unix time. The system then creates a streaming object for every channel of Labchart to be streamed. Next, the system registers an event called OnNewSamples which every 50 ms or so, is triggered when new samples are detected. This prompts a callback function (there is an independent callback function for every streaming object). In the callback function, a time offset array is created based on the number of new samples available and the sample rate. The first element of the time offset array in the current callback function is based on the last element in the previous callback. Each element of the time offset array is then added to the initial Unix timestamp taken at the beginning of the application to create the absolute Unix time of when the sample was collected. The timestamp, the data, and the source are then communicated via HTTP POST into InfluxDB. This continues until Labchart stops streaming or the application is terminated.

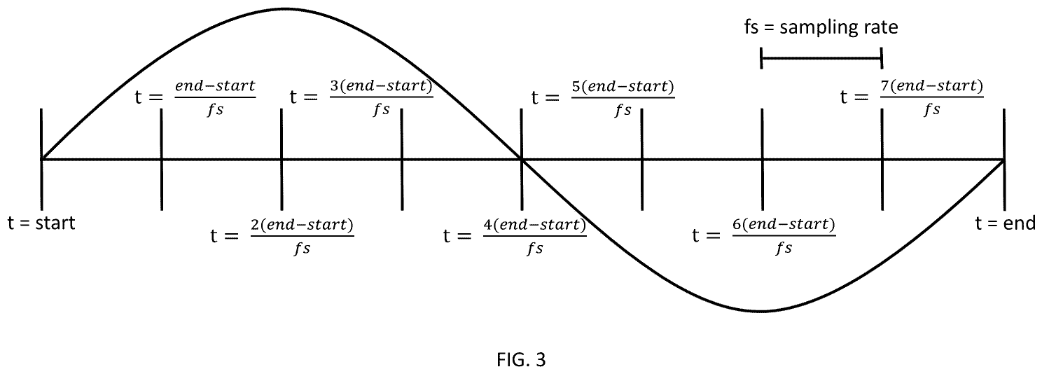

Referring to FIG. 3, an illustrative schematic is provided as to how evenly demarcated timestamps are generated if timestamps are not given by the devices. A period defined by start and end is divided into a number of equally spaced apart divisions based on a predetermined sampling rate. The first such division is time at end minus time at start divided by the sample rate (i.e., sampling frequency). The second division is simply twice the first division, and so on.

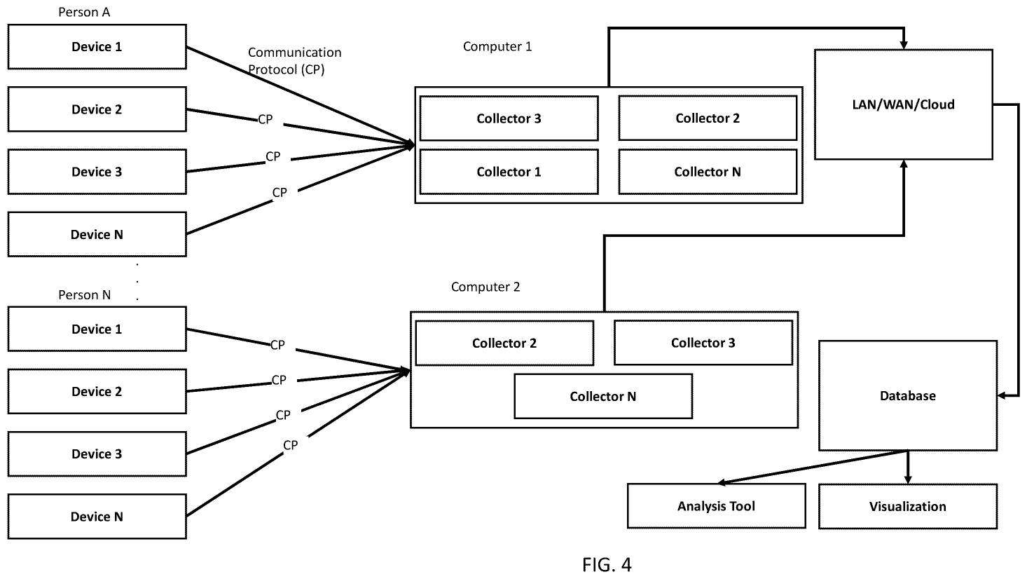

As for a network of communication between the system and the various devices, a generic network schematic is shown in FIG. 4. There can be any number of people's data streaming at once. If they are streaming at the same time, each person needs to have their own individual devices. The data may be indexed for each person; therefore, data of each person is encrypted. Next, each individual subject can have any number of biosignal measurement devices attached. These devices can be of any communication protocol. The dashed and solid lines represent that connections can be of various forms such as WiFi, Bluetooth, or USB. Each device has its own collector application. The overarching purpose of the collector application is to collect samples from its respective device and transmit those samples to the database. According to one embodiment, the collector applications take on average 5-10%. To avoid CPU over usage, collector applications can be connected to different computers, or even be run on a server in the cloud. The only requirement for what the collector applications are run on is that the computation device be able to connect to the database in the cloud. It is arbitrary what computer the collector applications are on because all the computers serve the same purpose of transmitting the data to the database in the cloud. This is represented by device one of person N being connected to computer 1. Regardless, all the data is still communicated with the database. Finally, the output layer (visualization and analysis) can be on any machine, including a local machine of the system. The output layer also only requires connection to the database. Having the database and output layers on a different machine may lead to less CPU usage.

According to one embodiment, the Equivital Vest, Varjo XR-3, and Powerlab 16/35 are all connected via USB. The E4 smartwatch is connected via a USB dongle (BLED 112) that plugs into the computer which connects to the watch using Bluetooth Low Energy. Their respective collectors (for the Varjo there is a Unity application and a native SDK application) are all run locally on an Alienware Workstation with an AMD RYZEN 9 5950x 16-Core Processor running on MICROSOFT Windows 10 Pro. Here, the information is sent to InfluxDB, a temporal database, using a HTTP POST request with LAN protocol. Finally, Grafana, running on the local machine, queries the database and graphs it in near real time.

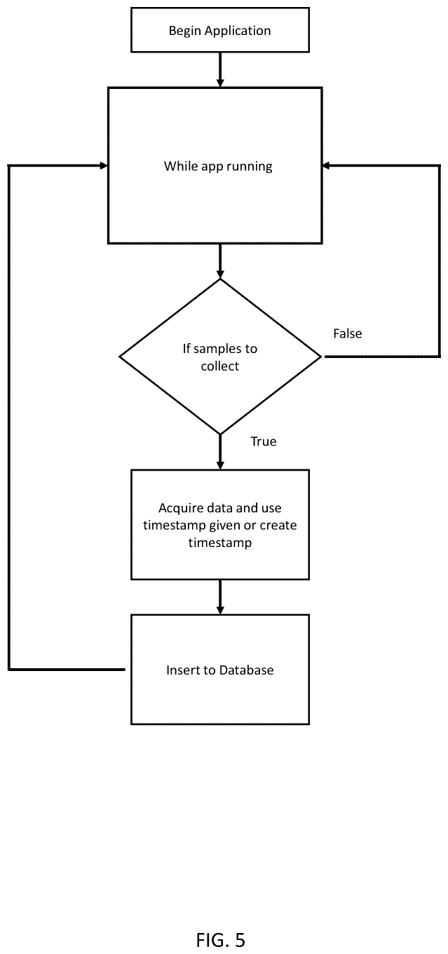

FIG. 5 is a flowchart of a general collector control flow that can be implemented to collect samples and timestamps. In this figure, if there are samples to be collected, the data is acquired and attached to a timestamp as discussed above. The combination of data and the timestamp is then stored in the database.

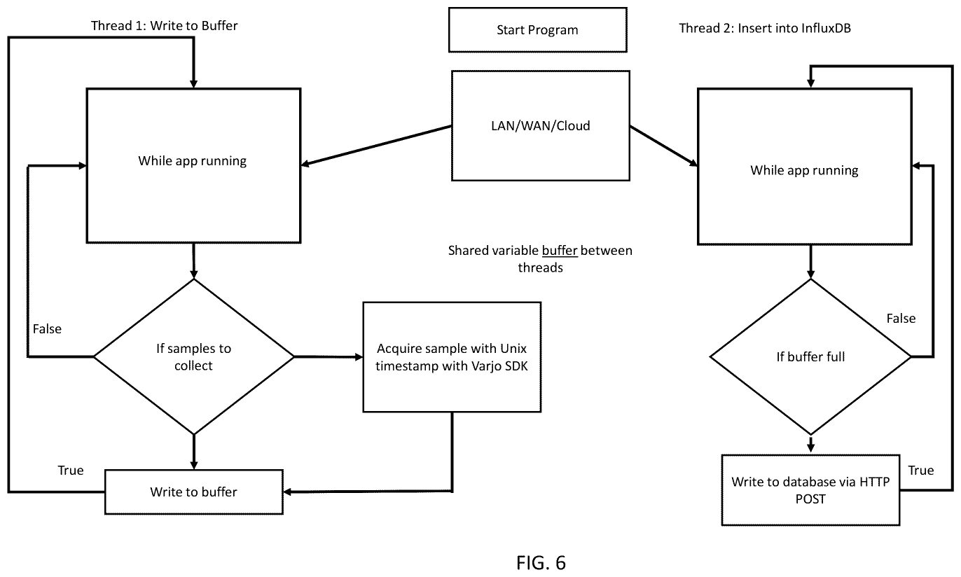

FIG. 6 is a flowchart depicting the control flow for the Varjo XR-3 headset to collect samples and timestamps. In this figure, there are two threads: 1) write to buffer; and 2) insert into the database. Depending on which thread, the data is acquired and placed either in a buffer or the database. This placement is continued until either the buffer or the database is full.

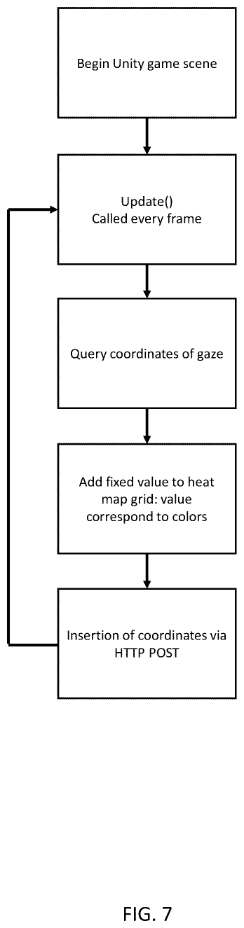

FIG. 7 is a flowchart depicting the control flow of how the heat map works in Unity for the Varjo XR-3 headset.

FIG. 8 is a flowchart depicting a collector layer, according to one embodiment, for any device that interfaces with the Labchart server to collect samples.

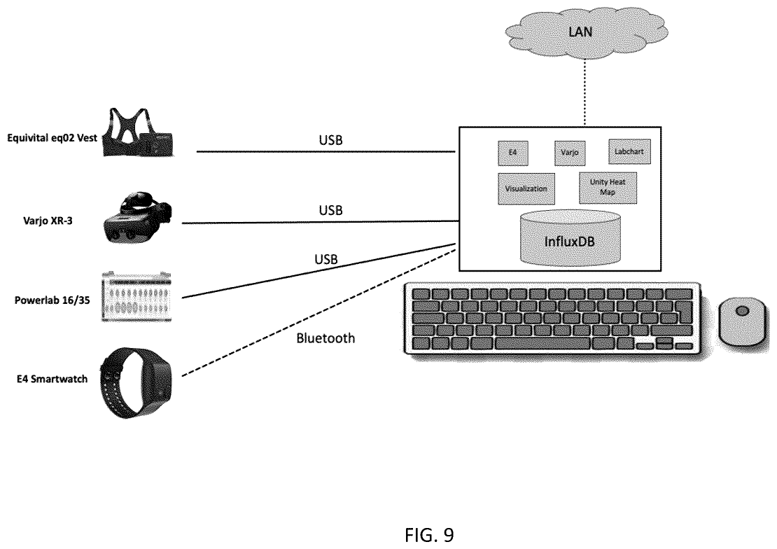

FIG. 9 is another schematic showing how devices are networked in the system of the present disclosure.

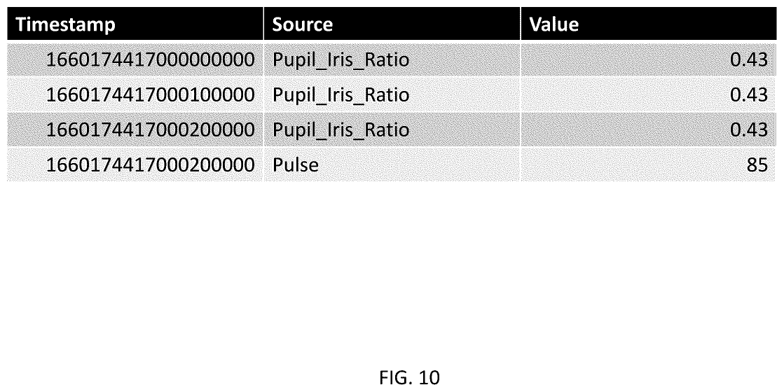

FIG. 10 is table showing sample entries into the implemented embodiment's database InfluxDB (see FIG. 6).

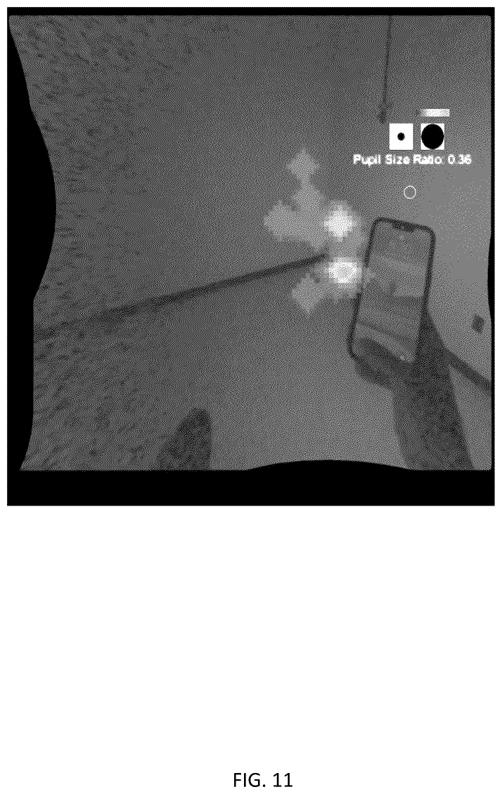

FIG. 11 is a heatmap depicting the augmented reality view inside the Varjo XR-3 headset with the heatmap tracking where the subject is looking at and gaze time(s), also implementable in virtual reality. At the top right, is a scale to show the length of time the user is looking in a spot: red means a shorter gaze and green means a longer gaze. Also, the circles demonstrate a real time proportional representation of the pupil dilation: a larger circle meaning the pupil is dilated and a smaller circle meaning the pupil is constricted.

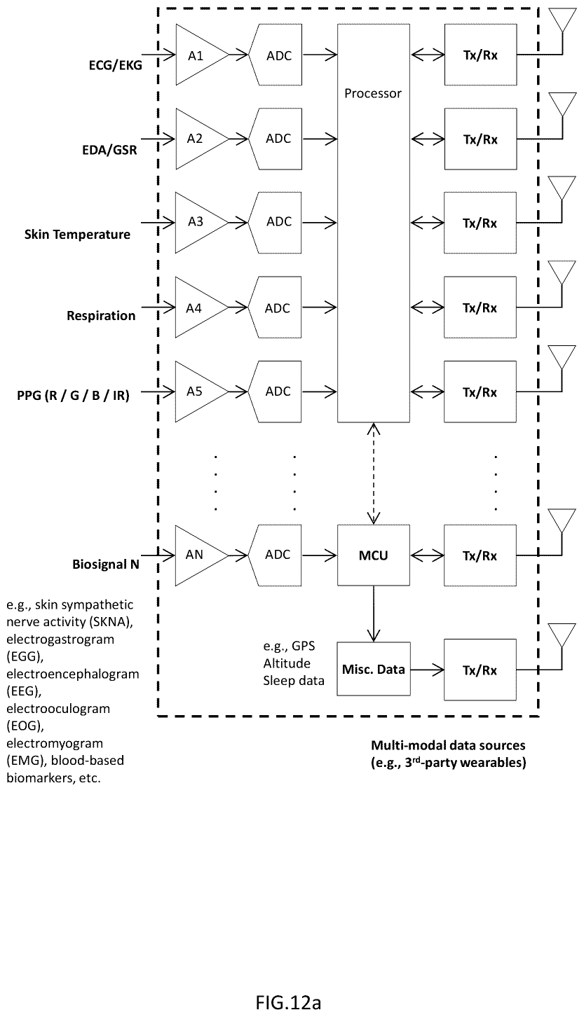

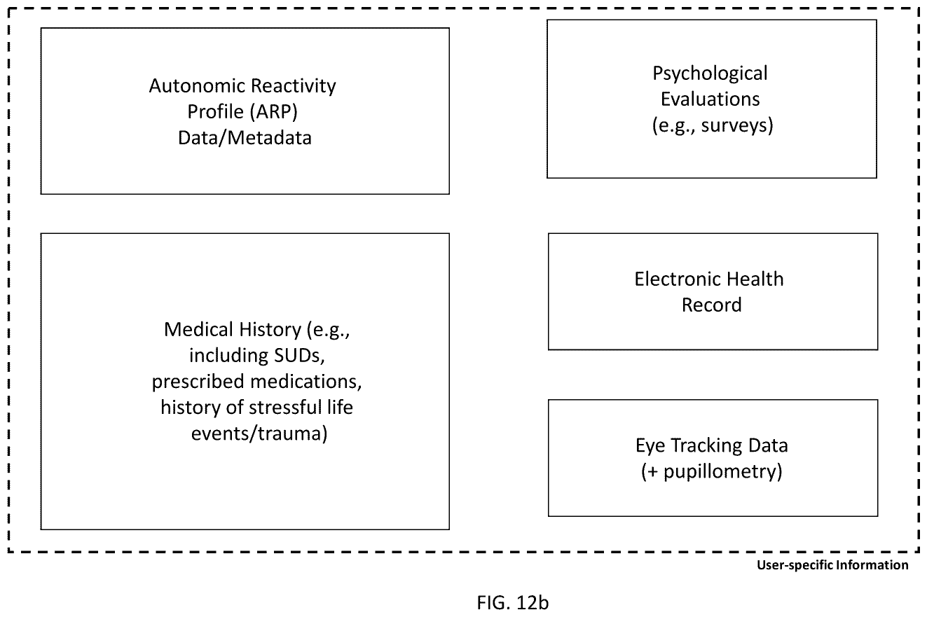

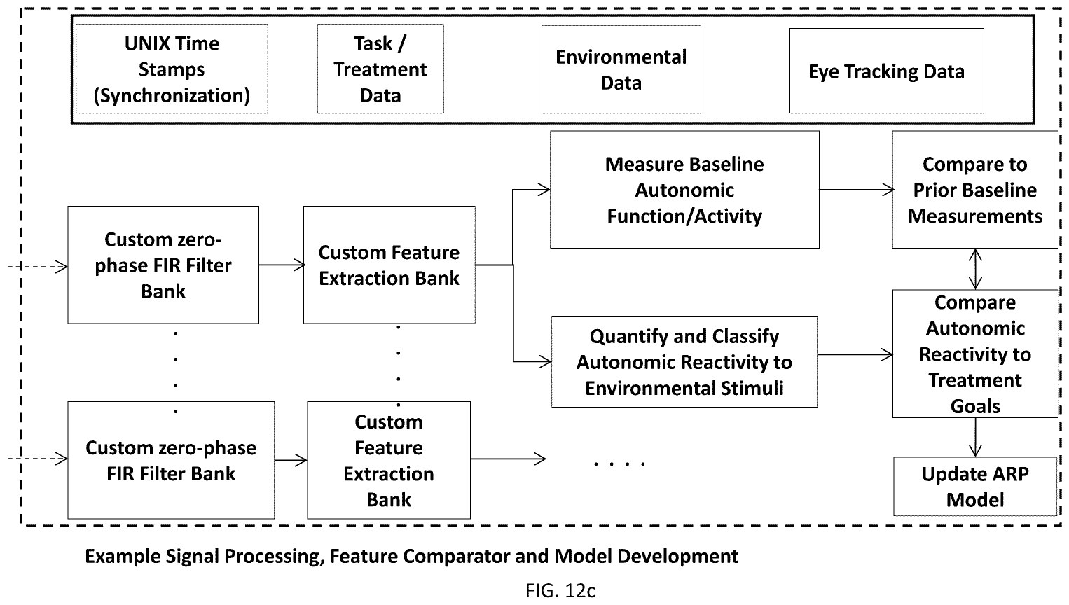

Referring to FIGS. 12a, 12b, and 12c, descriptive overview flowcharts are provided of the different types of biosignals alluded to herein and how the data associated with these bio-signals are transferred from the biosensors (e.g., wearables, etc.) to a central database and finally to a point where they can be used in a personalized model (referred to as the “Autonomic Reactivity Profile,” or ARP).

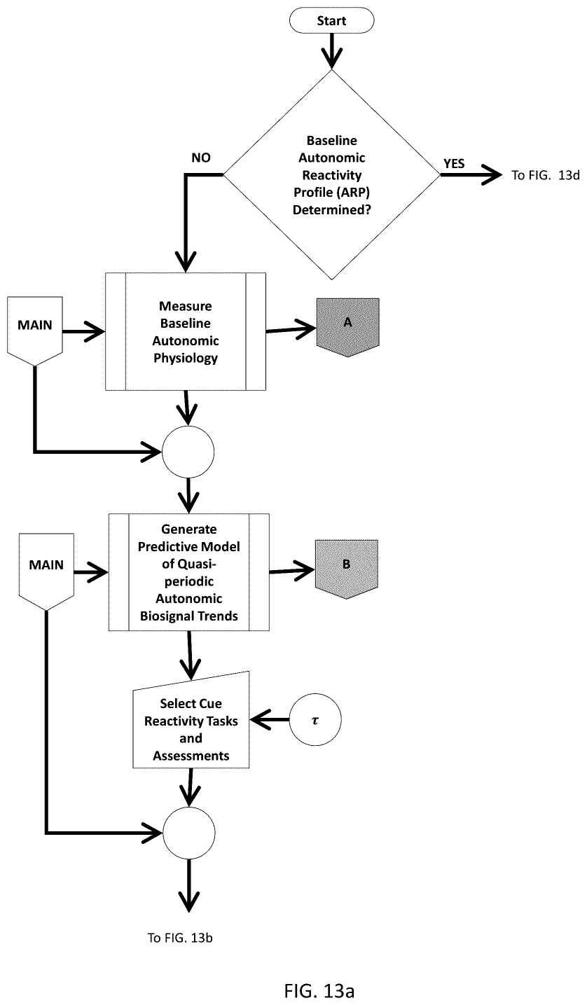

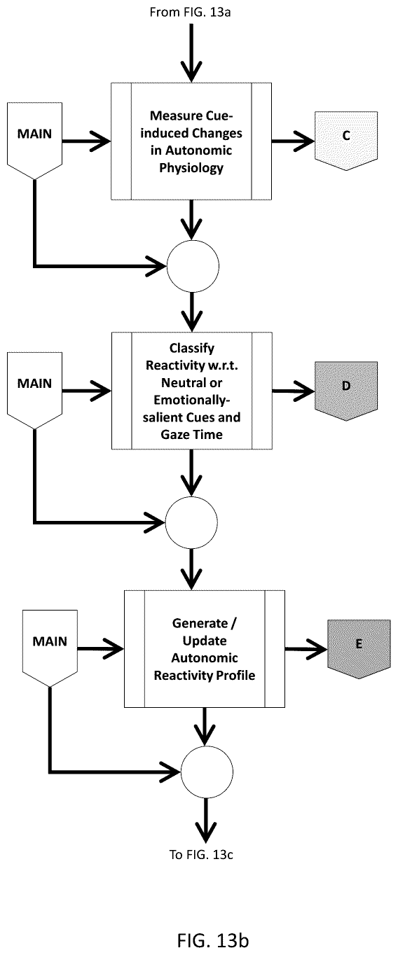

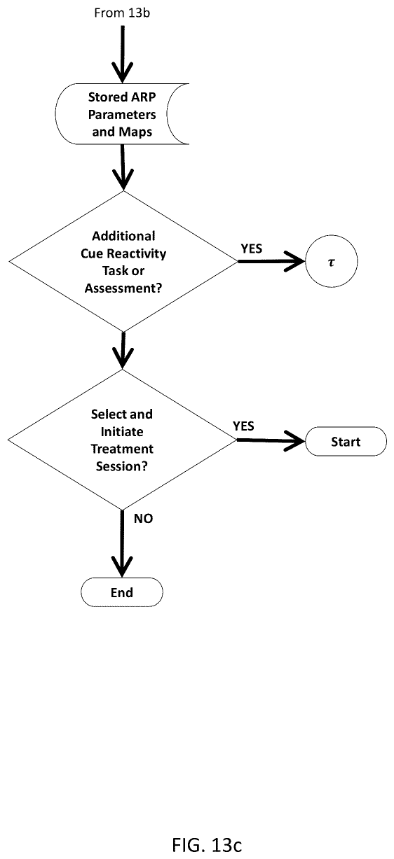

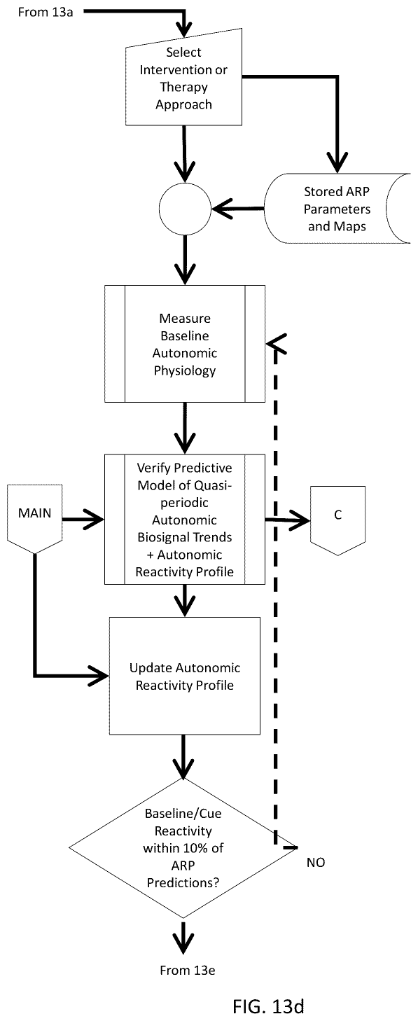

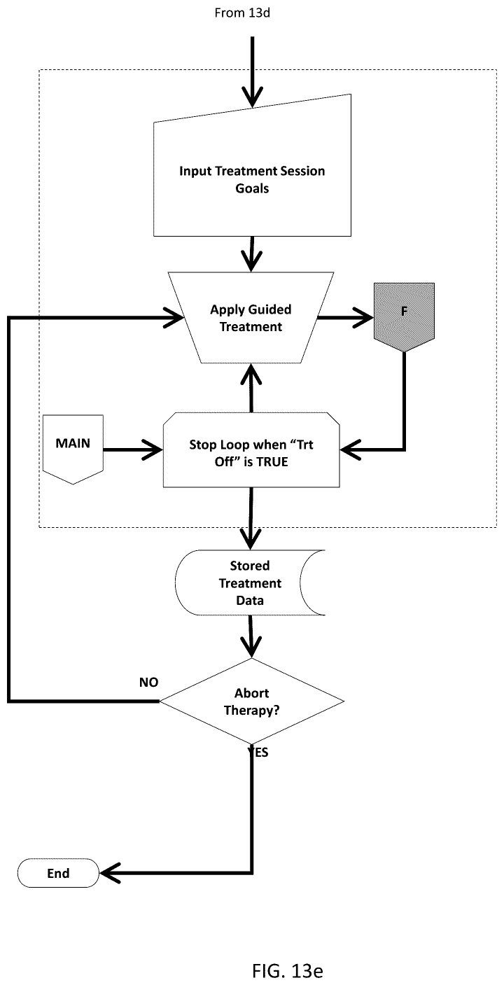

Referring to FIGS. 13a, 13b, 13c, 13d, and 13e a flowchart is depicted spanning across 5 pages. The flowchart in these figures provide a high-level process flow that describes the model training sequence and therapeutic/treatment sequence for a generalized application of the present system and method to treat anxiety disorders, substance use disorder(s), etc.

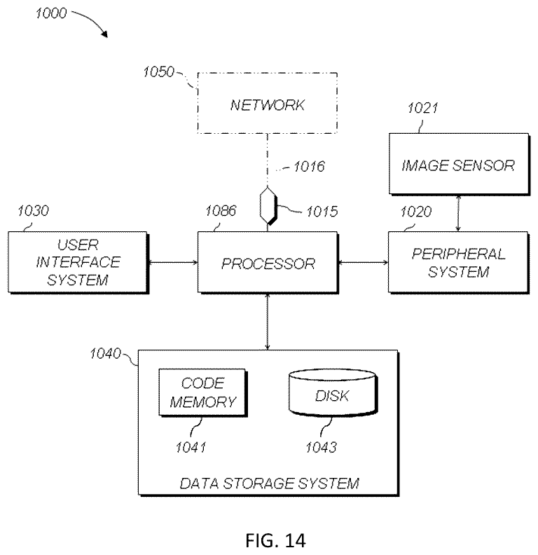

Referring to FIG. 14, an example of a computer system is provided that can interface with the above-discussed system. Referring to FIG. 14, a high-level diagram showing the components of an exemplary data-processing system 1000 for analyzing data and performing other analyses described herein, and related components. The system includes a processor 1086, a peripheral system 1020, a user interface system 1030, and a data storage system 1040. The peripheral system 1020, the user interface system 1030 and the data storage system 1040 are communicatively connected to the processor 1086. Processor 1086 can be communicatively connected to network 1050 (shown in phantom), e.g., the Internet or a leased line, as discussed below. The imaging described in the present disclosure may be obtained using imaging sensors 1021 and/or displayed using display units (included in user interface system 1030) which can each include one or more of systems 1086, 1020, 1030, 1040, and can each connect to one or more network(s) 1050. Processor 1086, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), or digital signal processors (DSPs).

Processor 1086 can implement processes of various aspects described herein. Processor 1086 can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise. Processor 1086 can include Harvard-architecture components, modified-Harvard-architecture components, or Von-Neumann-architecture components.

The phrase “communicatively connected” includes any type of connection, wired or wireless, for communicating data between devices or processors. These devices or processors can be located in physical proximity or not. For example, subsystems such as peripheral system 1020, user interface system 1030, and data storage system 1040 are shown separately from the data processing system 1086 but can be stored completely or partially within the data processing system 1086.

The peripheral system 1020 can include one or more devices configured to provide digital content records to the processor 1086. For example, the peripheral system 1020 can include digital still cameras, digital video cameras, cellular phones, or other data processors. The processor 1086, upon receipt of digital content records from a device in the peripheral system 1020, can store such digital content records in the data storage system 1040.

The user interface system 1030 can include a mouse, a keyboard, another computer (connected, e.g., via a network or a null-modem cable), or any device or combination of devices from which data is input to the processor 1086. The user interface system 1030 also can include a display device, a processor-accessible memory, or any device or combination of devices to which data is output by the processor 1086. The user interface system 1030 and the data storage system 1040 can share a processor-accessible memory.

In various aspects, processor 1086 includes or is connected to communication interface 1015 that is coupled via network link 1016 (shown in phantom) to network 1050. For example, communication interface 1015 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WiFi or GSM. Communication interface 1015 sends and receives electrical, electromagnetic or optical signals that carry digital or analog data streams representing various types of information across network link 1016 to network 1050. Network link 1016 can be connected to network 1050 via a switch, gateway, hub, router, or other networking device.

Processor 1086 can send messages and receive data, including program code, through network 1050, network link 1016 and communication interface 1015. For example, a server can store requested code for an application program (e.g., a JAVA applet) on a tangible non-volatile computer-readable storage medium to which it is connected. The server can retrieve the code from the medium and transmit it through network 1050 to communication interface 1015. The received code can be executed by processor 1086 as it is received, or stored in data storage system 1040 for later execution.

Data storage system 1040 can include or be communicatively connected with one or more processor-accessible memories configured to store information. The memories can be, e.g., within a chassis or as parts of a distributed system. The phrase “processor-accessible memory” is intended to include any data storage device to or from which processor 1086 can transfer data (using appropriate components of peripheral system 1020), whether volatile or nonvolatile; removable or fixed; electronic, magnetic, optical, chemical, mechanical, or otherwise. Exemplary processor-accessible memories include but are not limited to: registers, floppy disks, hard disks, tapes, bar codes, Compact Discs, DVDs, read-only memories (ROM), erasable programmable read-only memories (EPROM, EEPROM, or Flash), and random-access memories (RAMs). One of the processor-accessible memories in the data storage system 1040 can be a tangible non-transitory computer-readable storage medium, i.e., a non-transitory device or article of manufacture that participates in storing instructions that can be provided to processor 1086 for execution.

In an example, data storage system 1040 includes code memory 1041, e.g., a RAM, and disk 1043, e.g., a tangible computer-readable rotational storage device such as a hard drive. Computer program instructions are read into code memory 1041 from disk 1043. Processor 1086 then executes one or more sequences of the computer program instructions loaded into code memory 1041, as a result performing process steps described herein. In this way, processor 1086 carries out a computer implemented process. For example, steps of methods described herein, blocks of the flowchart illustrations or block diagrams herein, and combinations of those, can be implemented by computer program instructions. Code memory 1041 can also store data, or can store only code.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects. These aspects can all generally be referred to herein as a “service,” “circuit,” “circuitry,” “module,” or “system.”

Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into processor 1086 (and possibly also other processors), to cause functions, acts, or operational steps of various aspects herein to be performed by the processor 1086 (or other processors). Computer program code for carrying out operations for various aspects described herein may be written in any combination of one or more programming language(s), and can be loaded from disk 1043 into code memory 1041 for execution. The program code may execute, e.g., entirely on processor 1086, partly on processor 1086 and partly on a remote computer connected to network 1050, or entirely on the remote computer.

Those having ordinary skill in the art will recognize that numerous modifications can be made to the specific implementations described above. The implementations should not be limited to the particular limitations described. Other implementations may be possible.

1. A system for integrating a plurality of biosensor devices, comprising:
a plurality of biosensor devices connected to a subject;
one or more communication interfaces adapted to communicate with each of the plurality of biosensor devices;
a processor executing software on a non-transitory memory, the execution of the software configures the processor to:
establish a communication link with each of the plurality of biosensor devices each through the one or more communication interfaces;
receive biodata from each of the plurality of biosensor devices through the one or more communication interfaces at a sampling rate;
assign a timestamp to the received biodata such that the received data from the plurality of biosensor devices are synchronized;
receive environmental data via one or more environmental sensors;
store received biodata along with the assigned timestamp into a database and the environmental data;
analyze stored data along with the assigned timestamp to predict an evoked response to one or more stimuli and the environmental data; and
provide a feedback to the subject or a healthcare worker based on the analysis.

2. The system of claim 1, wherein the timestamp is provided by the plurality of the biosensor devices.

3. The system of claim 1, wherein the timestamp is generated based on a predetermined period divided by the sampling rate.

4. The system of claim 1, wherein the plurality of biosensor devices includes a smartdevice.

5. The system of claim 4, wherein the smartdevice provides heart rate biodata.

6. The system of claim 4, wherein the smartdevice provides electrocardiogram biodata.

7. The system of claim 4, wherein the smartdevice provides blood oxygen saturation biodata.

8. The system of claim 4, wherein the smartdevice provides blood pressure biodata.

9. The system of claim 4, wherein the smartdevice provides caloric expenditure biodata.

10. The system of claim 4, wherein the smartdevice provides sleep pattern biodata.

11. The system of claim 4, wherein the smartdevice provides perspiration biodata.

12. The system of claim 4, wherein the smartdevice provides position information of the subject.

13. The system of claim 12, wherein the environmental data includes geographical locations.

14. The system of claim 1, wherein the plurality of biosensor devices includes one or more of intelligent devices including virtual reality (VR) headsets, smart glasses, and artificial intelligent devices.

15. The system of claim 14, wherein the one or more intelligent devices provide heatmap biodata associated with where the subject is staring.

16. The system of claim 14, wherein the one or more intelligent devices provide eye movement biodata.

17. The system of claim 1, wherein the plurality of biosensors includes a continuous glucose monitoring device.

18. The system of claim 17, wherein the continuous glucose monitoring device provides glucose biodata.

19. The system of claim 1, wherein the plurality of biosensors includes a muscle contraction measurement device.

20. The system of claim 19, wherein the muscle contraction measurement device provides muscle contraction biodata.

21. The system of claim 1, wherein the received data is based on a synchronous data transfer protocol.

22. The system of claim 1, wherein the storing of the data is based on using a direct memory access protocol.

23. The system of claim 1, wherein the one or more communication interfaces include wireless channels.

24. The system of claim 1, wherein the one or more communication interfaces are wired.

25. The system of claim 23, wherein the wireless channels are based on Bluetooth connectivity.

26. The system of claim 23, wherein the wireless channels are based on Wi-Fi connectivity.
